(19) United States
(12) Patent Application Publication
(10) Pub. No.: US 2024/0328944 A1
(43) Pub. Date: Oct. 3, 2024

(54) Quantum Sensing of Paramagnetic Species Based on Nanodiamonds

(71) Applicant: Adamas Nanotechnologies Inc, Raleigh, NC (US)

(72) Inventors: Marco Diego Torelli, Raleigh, NC (US); Olga Aleksandrovna Shenderova, Raleigh, NC (US)

(21) Appl. No.: 18/583,400

(22) Filed: Feb. 21, 2024

Related U.S. Application Data
(63) Continuation-in-part of application No. 17/233,843, filed on Apr. 19, 2021, now Pat. No. 12,031,072.
Application No. 17/233,843 is a continuation of application No. PCT/US19/56986, filed on Oct. 18, 2019.
(60) Provisional application No. 62/747,700, filed on Oct. 19, 2018.

(51) Int. Cl. G01N 21/64

(52) U.S. Cl. CPC G01N 21/6428; G01N 33/389; G01N 2021/6439

(57) ABSTRACT

The present invention relates to a device and method of detection of paramagnetic chemical species by analyzing changes in a magnetically induced fluorescence contrast of fluorescent nanodiamond particles introduced into a sample.

This application is a Continuation in Part of U.S. application Ser. No. 17/233,843 filed on Apr. 19, 2021, which claims priority to PCT application number PCT/US19/56986 filed on Oct. 18, 2019 and U.S. provisional application No. 62/747,700 filed on Oct. 19, 2018 and are all incorporated herein in their entirety by reference.

This invention was made with government support under DE-SC0022441 awarded by U.S. Department of Energy Office of Science and 1R43GM144026-01 awarded by Department of Health and Human Services, National Institutes of Health, National Institute of General Medical Sciences. The government has certain rights in the invention. A portion of the research relating to the present technology was not federally sponsored.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a device and method of detection of paramagnetic chemical species by analyzing changes in a magnetically induced fluorescence contrast of fluorescent nanodiamond particles introduced into a sample.

### Description of Related Art

Nanodiamond (ND) particles containing nitrogen-vacancy (NV) centers (NDNV) exhibit uniquely coupled magneto-optical properties. The intensity of ND fluorescence depends on the NV centers' electronic states, which can be manipulated with external magnetic or electromagnetic fields. This quantum control and optical readout of the electronic state of the NV center in a combination with exceptional photostability and biocompatibility make NDNV a uniquely sensitive probe with applications from physics to biomedicine. One particular application of NDNV particles is detection of paramagnetic species including, but not limited to, reactive oxygen species (ROS), ferritin, gadolinium, manganese, and other species using the approach of NV T1 relaxometry. NV T1 relaxometry relates changes in NV relaxation time T1 (spin depolarization time) to the presence of magnetic noise (e.g., paramagnetic species) external to NV. Use of NV T1 relaxometry addresses problems with current optical reagents for ROS detection, such as organic fluorescent probes struggling with variability in their reporting primarily attributed to photobleaching and autocatalytic activation. This means that the traditional ROS probes do not accurately report on their targets and as such wastes time and resources. In practical implementation, NV T1 relaxation technology is based on application of microsecond light pulses to probe fluorescence during the spin relaxation of individual NDNV particles distributed in intra-cellular or extra-cellular environments and is most suitable for high-resolution microscopy. One limitation of NV T1 relaxometry is that it does not inherently address the problem of high fluorescence background which is often present in biological samples (e.g., in whole blood). Previously, it was shown that application of a periodically modulated magnetic field inducing periodically modulated ND-NV fluorescence is an effective way to separate the NDNV fluorescence signal from background autofluorescence. The use of phase-sensitive detection, such as that by lock-in amplifiers—a widely used signal processing method to extract small periodic signals present below noise levels—resulted in a further significant increase in the signal-to-noise ratio (SNR). Magnetically-modulated fluorescence has been used to perform NDNV imaging in the presence of high fluorescent background in different imaging scenarios, and, for example, demonstrated an impressive 100-fold improvement in SNR for NDNV labeling of sentinel lymph node. The readout of lateral flow assay (LFA) was recently improved by modulating fluorescence of ND-NV reporters associated (bound) with targeted analytes using microwaves and magnetic field in combination with using lock-in/narrow bandwidth signal amplification. All these applications of periodic magnetic modulation of NDNV fluorescence in combination with phase-sensitive detection were aimed to either image NDNV particles or enhance quantification of NDNV in the presence of fluorescence background.

### BRIEF SUMMARY OF THE INVENTION

The innovation of the present invention is in using magnetically induced fluorescence contrast ideally in combination with phase-sensitive detection to detect surrounding paramagnetic species. Moreover, to further improve the sensitivity of phase-sensitive detection, applicants implemented a double lock-in by adding amplitude modulation of the excitation light to the magnetic field modulation. This further reduced noise arising from instability in the light intensity, detector, or environmental noise.

In one embodiment, present invention relates to a method of monitoring of the fluorescence intensity of a diamond particle comprising:
a. taking plurality of diamond particles capable of exhibiting fluorescence in spectral ranges of about 640 nm to about 800 nm;
b. exciting fluorescence of the particles by optical radiation, and determining fluorescence intensity;
c. applying a magnetic field and optical radiation to the particles and determining fluorescence intensity; and
d. comparing the fluorescence intensity of b) with the fluorescence intensity of c).

In another embodiment the invention relates to a method of analyzing a paramagnetic chemical species comprising:
a. introducing diamond particles capable of exhibiting fluorescence containing at least a single NV center to a sample that may contain a paramagnetic chemical species to be analyzed and forming an analyzed mixture;
b. first applying optical radiation to the analyzed mixture, and detecting the resulting fluorescence intensity using a detector;
c. second, after the fluorescence intensity is detected, applying optical radiation, and applying a magnetic field to the analyzed mixture and detecting the resulting fluorescence intensity using a detector;
d. comparing detected fluorescence intensity in (b) and (c), to provide a fluorescence contrast; and
e. using the fluorescence contrast, determining the presence of paramagnetic chemical species.

In another embodiment, it relates to a method of detection of a chemical species comprising:
a. introducing diamond particles capable of exhibiting fluorescence containing at least a single NV center to a sample that may contain the chemical species to be analyzed and forming an analyzed mixture;
b. first applying optical radiation with periodically time-varying intensity to the analyzed mixture using a light source, and detecting the resulting time-varying fluorescence intensity using a detector;
c. second, after the first fluorescence intensity is detected, applying optical radiation with periodically time-varying intensity and a magnetic field to the analyzed mixture and detecting the resulting time-varying fluorescence intensity using a detector,
d. processing the detected fluorescence intensity in (b) and (c) using a phase-sensitive detection and comparing detected fluorescence intensity in (b) and (c), providing fluorescence contrast; and
e. determining the presence of the chemical species from the fluorescent contrast.

In another embodiment it relates to a method of detection of a chemical species comprising:
a. introducing diamond particles capable of exhibiting fluorescence containing at least a single NV center to a sample that may contain the chemical species to be analyzed and forming an analyzed mixture;
b. applying optical radiation with periodically time-varying intensity and also applying a magnetic field with periodically time-varying magnitude to the analyzed mixture with a magnetic modulation frequency selected from about 1 Hz to about 10,000 Hz, and detecting the resulting time-varying fluorescence intensity using a detector;
c. processing the detected time-varying fluorescence intensity in (b) using a phase-sensitive detection, providing magnetically modulated fluorescence contrast; and
d. using the fluorescence contrast, determining the presence of chemical species.

In yet another embodiment it relates to a device for detection of a chemical species comprising:
a. a container having a sample that contains an analyzed mixture that may contain the chemical species and fluorescent diamond particles containing at least a single NV center;
b. a light source for applying optical radiation with periodically time-varying intensity at an optical modulation frequency selected from about 10 Hz to about 10,000 Hz to the analyzed mixture;
c. a source generating a magnetic field for applying to the analyzed mixture at a magnetic modulation frequency selected from about 1 Hz to about 10,000 Hz;
d. a detector detecting fluorescence intensity from the analyzed mixture, where the fluorescence intensity is optically modulated, or optically and magnetically modulated; and
e. a signal processing device providing a phase-sensitive measurement of a detected fluorescence intensity modulated at two frequencies, where the detected fluorescence intensity is processed by at least one of: a dual-frequency lock-in amplifier, two individual lock-in amplifiers, digital lock-in analysis, computational lock-in analysis, Fourier analysis, analog-to-digital converter, and computer analyzer.

### BRIEF DESCRIPTION OF THE DRAWINGS

FIG. 1 is an illustration of dual-frequency signal modulation block diagram schematically showing components for detection of analyte by magnetic modulation of nanodiamond fluorescence.

FIG. 2A is an illustration of an unmodulated NDNV signal.

FIG. 2B is an illustration of magnetically modulated NDNV signal.

FIG. 2C is an illustration of optically modulated NDNV signal.

FIG. 2D is an illustration of simultaneous optically and magnetically modulated NDNV signal.

FIG. 3A is an illustration of noise in raw NDNV signal.

FIG. 3B is an illustration of significant noise reduction of nanodiamond fluorescence signal by using optical modulation of excitation light.

FIG. 3C is an illustration of differentiation of analyte mixtures 1 and analyte mixture 2 as shown by different voltage outputs generated in the presence or absence of a static magnetic field.

FIG. 4. is an illustration of the effect of external magnetic field on normalized fluorescence contrast.

FIG. 5A is an illustration of measurement of stable radicals an analyzed mixture of TEMPOL.

FIG. 5B is an illustration of measurement of stable radicals an analyzed mixture of TEMPOL amine.

FIG. 5C is an illustration of the measurement and differentiation of nitroxide functionalized diamond differentiated from diamond without nitroxide functionalization.

FIG. 6 is an illustration of measurement of paramagnetic Gd ions in an analyzed mixture.

FIG. 7 is an illustration of differentiation of analytes containing ferritin or apoferritin.

FIG. 8 is an illustration of measurement of ROS produced in an enzymatic reaction.

FIG. 9A is an illustration of measurement of peroxide in an analyzed mixture by total optical signal with and without magnetic field.

FIG. 9B is an illustration of measurement of peroxide in an analyzed mixture by normalized fluorescence contrast over time before and after peroxide addition.

FIG. 10A is an illustration of the variability in spectra with magnetic field on and off without normalization.

FIG. 10B is an illustration of the noise reduction due to normalization of NDNV emission intensity to unchanged fluorescence intensity of NVN centers with magnetic field on and off.

FIG. 10C is an illustration of noise in wavelength-dependent modulation measured in the presence of iron (III).

FIG. 10D is an illustration of reduced noise in wavelength-dependent modulation measured in the presence of iron (III) by normalization to NVN emission.

FIG. 11 is an illustration of the measurement of solvent deoxygenation and oxygenation.

FIG. 12A is an illustration of the detection of an analyte by fluorescent nanodiamond particles without silica shell.

FIG. 12B is an illustration of the detection of an analyte by fluorescent nanodiamond particles with a very thin silica shell under 1 nm.

FIG. 12C is an illustration of the detection of an analyte by fluorescent nanodiamond particles with a thin silica shell of several nanometers.

FIG. 13 is an illustration of differentiation of analytes containing free-radical TEMPOL and its diamagnetic analog.

FIG. 14A is an illustration of yeast targeted NDNV detected by ODMR for detection of analyte in cultured of yeast cells.

FIG. 14B is an illustration of non-targeted NDNV undetectable by ODMR in cultured of yeast cells.

FIG. 14C is an illustration of the detection of yeast-targeted NDNV by magnetically induced fluorescence contrast as compared to non-targeted NDNV.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publication may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “nanodiamond or diamond nanoparticles” refers to submicron sized particles. More specifically, the term nanodiamond particles refers to discrete diamond particles exhibiting at least one spatial dimension having a size of less than about 1000 nm. More commonly, the nanodiamond particles exhibit multiple spatial dimensions having a size of less than about 1000 nm.

As used herein, the term “micron-diamond or micron diamond particle” refers to discrete diamond particles exhibiting at least one spatial dimension having a size ranging from about 1 micron (μm) to 500 μm, while other dimensions are more than about 1 micron. More commonly, a micron diamond particle exhibits multiple spatial dimensions having a size of about 1 μm to 500 μm (0.5 mm).

As used herein, the term “FND” refers to fluorescent nanodiamond, a diamond nanoparticle exhibiting fluorescence.

As used herein, the term “luminescence” refers to the emission of electromagnetic radiation from crystallographic defects within the diamond lattice upon absorption of radiative energy from an appropriate excitation source of electromagnetic radiation.

As used herein, the term “electromagnetic radiation” refers to the propagating waves of electric and magnetic waves carrying radiative energy through space. More specifically, this term refers to the radiative energy necessary to promote luminescence emission from crystallographic defects centers within the diamond lattice, and may consist of ultraviolet, visible, infrared, or X-rays.

As used herein, the term “H3 color centers” refers to specific crystallographic defect centers within the diamond lattice which can exhibit luminescence. More specifically, the H3 center refers to the specific defect center consisting of an atomic arrangement of two nitrogen atoms surrounding a vacancy which can exhibit green luminescence upon excitation with an appropriate source of electromagnetic radiation.

As used herein, the term “quantum properties” of diamond particles is related to spin characteristics of the NV centers including, but not limited to, the spin coherence time (T2), spin dephasing time, spin-lattice relaxation time (T1), optically detected magnetic resonance (ODMR) spectroscopic characteristics (full width at half maximum, FWHM), ODMR contrast, and the difference in luminescence intensity of NV centers under applied magnetic field as compared to the luminescence intensity measurements without magnetic field. These characteristics can also include 13C dynamic nuclear polarization capability of P1 centers (single N dopants). Improvement in spin electronic and nuclear characteristics of color centers and spin-containing lattice elements is based on decrease of the lattice distortions and elimination of parasitic defects following high temperature annealing.

As used herein, the term “fluorescence intensity” means the rate of delivery of energy of photons emitted by analyzed mixture and measured, for example, by the signal from a photodiode.

As used herein, the term “diamond particle capable of exhibiting luminescence” means any discrete carbon-based material with a diamond cubic lattice which emits light via electronic excited transitions.

As used herein, the term “spectral range of 640 nm to 800 nm” means range of wavelengths of electromagnetic radiation emitted by NV centers in diamond.

As used herein, the term “optical radiation” means the optical radiation comprising wavelengths from the range about 190 nm to 2000 nm used for excitation of color centers in diamond.

As used herein, the term “magnetic field” means the vector magnetic field which exerts force on a magnetic body and moving electric charges.

As used herein, the term “sensing changes in the environment” means reporting or providing a means of observing the presence or change in the quantity of paramagnetic species, other species, temperature, and the like near the diamond particle.

As used herein, the term “paramagnetic chemical species” means a chemical analyte containing chemical species with one or more unpaired electrons. Where chemical species comprise: species, structures and molecules with unpaired electrons; free radicals, hydrogen peroxide, peroxides, paramagnetic metal ions, paramagnetic metal ion chelates, reactive oxygen species, reactive nitrogen species, reactive sulfur species, ferritin, metalloprotein, oxygen molecules, gaseous molecules containing unpaired electrons, gaseous molecules dissolved in fluid, spin traps, chemical traps; free radicals in proteins, antibodies, antigens; free radicals in nucleic acids; products of enzymatic reactions, products of metabolic reactions; free radicals in polymers.

As used herein, the term “NV center” means nitrogen-vacancy point defect present within the diamond lattice consisting of replacement of two adjacent carbons with a nearest neighbor pair of a nitrogen atom and lattice vacancy (site where a carbon atom is absent from the crystallographic lattice). NV center can have a neutral charge state or a negative charge state. In the majority of embodiments, the NV center as used herein is NV center with negative charge state.

As used herein, the term “analyzed mixture” means a combination of fluorescent nanodiamond particles containing NV centers and analyte, for example, a suspension containing fluorescent nanodiamonds and freely dispersed analyte molecules.

As used herein, the term “fluorescence detector” means any device capable of reporting fluorescence intensity or changes in the fluorescence intensity.

As used herein, the term “fluorescence signal detector” means a detector of fluorescence intensity.

As used herein, the term “fluorescence contrast” means difference in fluorescence intensities detected with and without applying magnetic field.

As used herein, the term “periodically time-varied light intensity” means repeated changes in the intensity of generated light over time, where light intensity is varied between low intensity state (or no light) and maximum intensity state with a certain frequency.

As used herein, the term “light source” means a device capable of producing photons including but not limited to the combination of the photon-generating device with means to produce light with periodically time-varying intensity or continuous radiation.

As used herein, the term “alternating in time” means repeated changes in the magnitude of a physical parameter (e.g. applied magnetic field) over time.

As used herein, the term NDNV is an abbreviation for nanodiamond particles containing nitrogen-vacancy (NV) centers.

As used herein, the term “phase-sensitive detection” refers to a method (lock-in detection) and an electrical instrument (lock-in amplifier or computer providing digital analysis of a recorded waveform) capable of extracting signal amplitudes and phases. A lock-in measurement extracts signals in a defined frequency band around the reference frequency, efficiently rejecting all other frequency components to report the signal magnitude.

As used herein, the term “modulating” refers to changing a physical parameter over time; where, for example, changing of magnetic field on and off can be a single event, or can be repeated multiple times with different time intervals between magnetic field on and off, or repeated in a periodic manner over time with a certain frequency.

As used herein, the term “nutritious substance” refers to food, the substance consisting essentially of protein, carbohydrate, fat, and other nutrients used in the body of an organism.

According to certain embodiments consistent with the present invention, diamond particles are introduced to a sample that may contain a paramagnetic chemical species to be analyzed forming an analyzed mixture, further comprising applying magnetic field for modulating fluorescence of NV centers and providing a fluorescence contrast; and where using the fluorescence contrast comprises determining the presence of paramagnetic chemical species. In another embodiment, fluorescent diamond particles contain plurality of NV and H3 color centers, where fluorescence intensity of NV centers measured with and without applying magnetic field is calibrated to the unchanged fluorescence intensity produced by excitation of H3 centers. This calibration improves sensitivity of the paramagnetic species detection.

According to certain embodiments consistent with the present invention, concentration of the fluorescent diamond particles in the analyzed mixture is known. It can be selected, for example, including but not limited to, from a range between about 1 ug/ml to about 10 mg/ml of NDNV. In one embodiment, presence of paramagnetic chemical species is defined by comparison between values of a fluorescence contrast in the analyzed mixture and a control sample, where the paramagnetic chemical species are absent. In another embodiment, concentration of paramagnetic chemical species in the analyzed mixture is defined from a calibration curve obtained from samples with known concentration of the paramagnetic chemical species. In yet another embodiment the fluorescence contrast is normalized by the value of fluorescence intensity detected in the state of no applied magnetic field compared to applied magnetic field, providing a normalized fluorescence contrast, so that the presence of paramagnetic chemical species is defined by comparison of values of a normalized fluorescence contrast in the analyzed mixture and a control mixture where the paramagnetic chemical species are absent. In another embodiment, the concentration of paramagnetic chemical species changes over time and thus, the presence of paramagnetic chemical species is defined from changes in the fluorescence contrast over time. Diamond particles used in the present invention comprise sizes between approximately 5 nm and 1000 nm, preferably between 10 nm and 200 nm, and most preferably between 20 nm and 70 nm.

In certain embodiments of the present invention diamond particles comprise particles with modified surface providing change in the fluorescence contrast compared to particles with unmodified surface comprising addition or removal of surface groups, including but not limited to surface spins, surface dangling bonds, surface charges, dipole-dipole interactions, and addition of groups or shells of differing dielectric composition from diamond such as silica. Modifying the diamond particle surface comprises functionalizing the diamond particles with at least one functional surface group selected from the group consisting of carboxylic, hydroxyl, amino, hydrogen, epoxy, poly(ethylene glycol), poly(glycerol), hydrocarbon, aromatic, nucleophile, thiol, sulfur, acid, base, silane, aluminum, halogen, and fluoro-containing. The modified diamond particle surface can further comprise changes in at least one of surface spins, surface dangling bonds, and surface charges due to the presence of analyte paramagnetic chemical species and where the change in the spin properties of the modified surface impacts spin properties of the NV centers and resulting fluorescence contrast. Changes in surface spins and surface charges of the modified surface could cause changes in the T1 relaxation time of NV centers resulting in a change in magnetically modulated fluorescence contrast. In another embodiment consistent with the present invention, diamond particles are modified with chemical groups which convert a transient analyte into stable forms. In yet another embodiment the analyzed mixture is altered to adjust the fluorescence contrast comprising at least one from the group comprising addition of non-analyte calibrants, chemical species, oxygenation, deoxygenation, diluted gaseous species, pH altering species and pH stabilizing species, salts. Yet another embodiment comprises conjugating with the diamond particles or attaching to the diamond particles at least one material selected from the group consisting of biological molecules, a site-specific targeting ligand, a nucleic acid, a peptide, a protein, an antibody, an antigen, oligonucleotide, aptamer, RNA, DNA, a ligand, a dye, a fluorescent specie, a spin trap, a radioactive specie, an image contrast agent, an isotope, a drug molecule, a hormone, a carbohydrate, and a polymer.

In certain embodiments of the present invention the analyzed mixture is produced by comprising one of: pouring, mixing, shaking, vortexing, incorporation by sonication, ballistic delivery of fluorescent nanodiamonds, ballistic delivery using a gene gun, drying of a mixed suspension, exposing a sample with analyte to fluorescent nanodiamond immobilized on a substrate, capillary, wall of a well, optical fiber or inside an optical fiber. In another embodiment the analyzed mixture further comprises: whole blood, blood plasma, serum, body fluids, fluids, nutritious substance, waste-water, environmental liquids, buffer, cellular membranes, intracellular membranes, cell compartments, organelles, cytoplasm, animal cell, stem cell, eukaryotic cell, prokaryotic cell, cell culture, intracellular fluid, organism, organ, tissue, plant fluids, plant tissue, plant cell, microorganism, bacteria, yeast, yeast membrane, yeast cytoplasm, intercellular fluid in a bioreactor, animal cell in a bioreactor, biomass in a bioreactor, products of fermentation, fermentation biomass, marine bacteria, phytoplankton, seaweeds, corals, microfluidic chip, organ-on-a chip, polymers, plastics, nitrocellulose membrane, optical fiber, matrix or a substrate, where the substrate is electronic component, a tag, a tracer, a label, a polymer, and an optically transparent solid.

In certain embodiments of the present invention analyzed mixture comprises at least one of: a continuous flow in a capillary or a channel, flow in a microfluidic device, and content of a multi-well plate. In another embodiment method of detection of a chemical species using magnetically modulated fluorescence of NVND further comprises a microfluidic flow assay, a microplate reader, flow cytometry assay, fluorescence activated cell sorting, an anti-Brownian electrokinetic (ABEL) trap, an acoustofluidic device, an electrophoretic device, a lateral flow assay, a vertical flow assay, PCR, and ELISA. For example, a possible method of detection of paramagnetic species using a lateral flow assay would comprise a method where:
a. fluorescent diamond particles are conjugated with a ligand that captures analyte;
b. fluorescent diamond particles with captured analyte are captured at a test line of a lateral flow strip; and
c. paramagnetic nature of analyte is concluded from analysis of the magnetically modulated fluorescence contrast of fluorescent diamond particles.

In one embodiment of the detection of paramagnetic species using a lateral flow assay the analyte further comprises ferritin or apoferritin.

In certain embodiments of the present invention applying a magnetic field to the analyzed mixture comprises applying a periodically time-varying magnetic field with known frequency, amplitude, phase, and waveform. In one embodiment, a magnetic modulation frequency comprises a frequency selected from a range between about 1 Hz to about 10,000 Hz, most preferably from about 10 Hz to about 1,000 Hz. The amplitude of the applied magnetic field, either static or periodically time-varying, can be varied between about zero Gauss to a maximum magnetic field or between a minimum and a maximum magnetic field. In one embodiment, maximum magnetic field comprises magnetic field strength between approximately 1 Gauss and 5,000 Gauss and most preferably between approximately 400 and 1000 Gauss.

In certain embodiments of the present invention the periodically time-varying light intensity is further characterized by a known frequency, amplitude, phase, and waveform. In one embodiment, an optical modulation frequency comprises frequency selected from about 10 Hz to about 10,000 Hz. The optical radiation has wavelength between approximately 480 nm and 1100 nm, preferably between 530 nm and 640 nm, and most preferably between 530 nm and 580 nm.

Innovation of the present invention is in using magnetically induced fluorescence contrast in combination with phase-sensitive detection to detect surrounding paramagnetic species. Moreover, to further improve the sensitivity of phase-sensitive detection, the method comprises a double lock-in by adding amplitude modulation of the excitation light to the magnetic field modulation. This further reduced noise arising from instability in the light intensity, detector, or environmental noise. One embodiment of the present invention comprises taking an analyzed mixture and applying optical radiation with periodically time-varying intensity providing optical modulation frequency and also applying a magnetic field with periodically time-varying magnitude providing magnetic modulation frequency. The method further comprises phase-sensitive measurement of the detected fluorescence signal originated from the analyzed mixture modulated at the optical and magnetic modulations frequencies, where the detected fluorescence signal is processed by at least one of: a dual-frequency lock-in amplifier, two individual lock-in amplifiers, digital lock-in, computational lock-in, and Fourier analysis. In one embodiment the method further comprises parallel processing of the signals from two lock-in amplifiers synchronized to the same oscillator. In another embodiment the detected resulting fluorescence intensity may be further analyzed by an analog-to-digital convertor, where the converter is selected from the group comprising of oscilloscope, data acquisition device, and lock-in amplifier.

### DRAWINGS

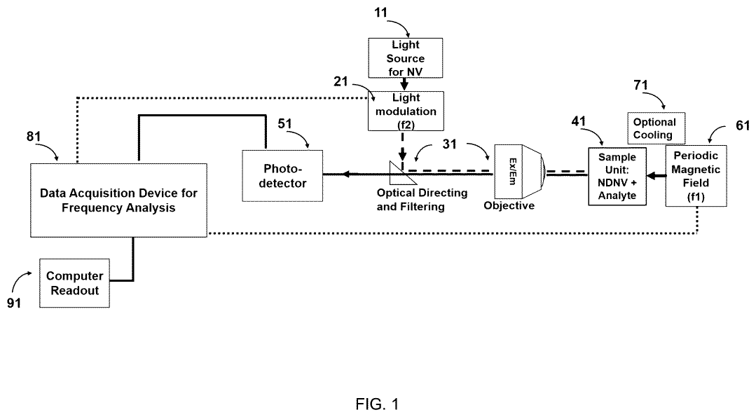

Now referring to the drawings, FIG. 1 is an illustration of a block diagram of components for implementation of detection of analyte by magnetic modulation of nanodiamond fluorescence. A light source (11) comprising a laser, LED, Hg lamp, or other suitable light source is periodically modulated at frequency f2 (21), either directly by using a pulsed light source or through external modulation of produced light (e.g. pulsed current, arbitrary wave generator, or related), which may be accomplished, for example, by an optical modulator (e.g. mechanical light chopper, acousto-optical modulator, electro-optical modulator, or similar device), or any device and structures known in the art which can provide optical radiation with periodically time-varying intensity. Light is directed and/or focused (31) to the sample unit (41), where a sample that may contain analyte is mixed with fluorescent diamond particles. Emission light from the sample unit is subjected to further optical filtering (31) and directed to a photodetector (51). Optical directing and filtering of excitation and emission light may occur in the same space or may be present in multiple, separated geometries (e.g. at 90 degrees; pass through; reflection, multiple beam splitters, multiple filters, etc). Optical modulation (21) may alternatively be applied to emission light before the photodetector (51) instead of or in addition to light modulation at the excitation source (11). The sample unit is exposed to a time-varying magnetic field (61) at frequency f1. The sample and accompanying magnetic field modulation unit may be optionally cooled (71). Multiple photodetectors (51) may be used with simultaneously or/and with different combinations/configurations of the optical directing unit (31), for example splitting excitation and/or emission light by multiple beamsplitters providing independent measurements. A detector for the fluorescence signal can be one of including, but not limited to: avalanche photodiode detector, single photon detector, charged coupled device, or photomultiplier tube. The output of the photodetector(s) (51) is read by a data acquisition device (81) for frequency analysis. Manifestations of the data acquisition device may consist of two lock-in amplifiers reporting independent signals, a single multifrequency lock-in amplifier, or direct measurement for frequency dependent analysis (e.g. Fourier transform or related methodologies). Magnetic modulation (61) and optical modulation (21) frequencies are communicated to a data acquisition device (81) to decode frequency-dependent data. Data is further analyzed, displayed, and recorded on a computer for readout and interpretation (91).

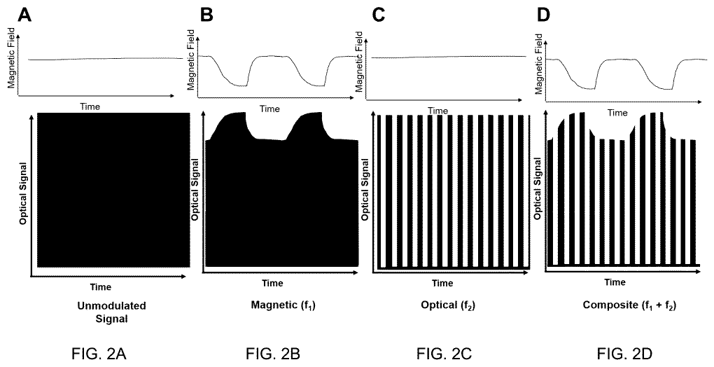

FIGS. 2A, 2B, 2C and 2D are illustrations of specific examples of the different fluorescence emission optical waveforms that may be generated by optical modulation (f2) and/or magnetic modulation (f1) and detected by a photodetector producing a corresponding output which may then be analyzed. Illustrated in FIG. 2A NDNV fluorescence in the absence of any modulation is constant over time. FIG. 2B is an example of magnetically modulated fluorescence where the magnetic field waveform reflects the inductive charge/discharge of an electromagnet. NDNV fluorescence is thus modulated with a periodicity and shape dependent on the oscillating magnetic field. The shape depends on the field generated by the magnet and may be produced via electromagnet(s), solenoid(s), Helmholtz coils, mechanically moving static magnetic units, moving magnetic shielding, or any combination of thereof. The orientation of the magnetic field (along x-, y- or z-axis) may be controlled using, for example, one, two or three Helmholtz coils, or any prior described means of field generation, arranged perpendicular to each other. Magnetic fields along each direction can be modulated independently at different frequencies, with constant or variable phase differences, and have different values of the magnetic field strength. FIG. 2C illustrates optical modulation that is used to reduce noise, including but not limited to variation in the intensity of excitation light produced by a light source, detector noise, electromagnetic environmental noise from surrounding equipment, and mechanical noise. Modulation may occur at the excitation light or emission light source and produces a response at the photodetector proportional to the reduction of the total light intensity due to light modulation. Shown is an example of fully attenuating modulation as a square-wave similar to that of a mechanical optical chopper when light is periodically completely blocked. FIG. 2D illustrates an example of the summed waveform of optical modulation (f2, FIG. 2C) and magnetic modulation (f1, FIG. 2B). The composite signal contains both modulations (f1+f2) and is extracted through phase-sensitive detection, for example through a lock-in amplifier.

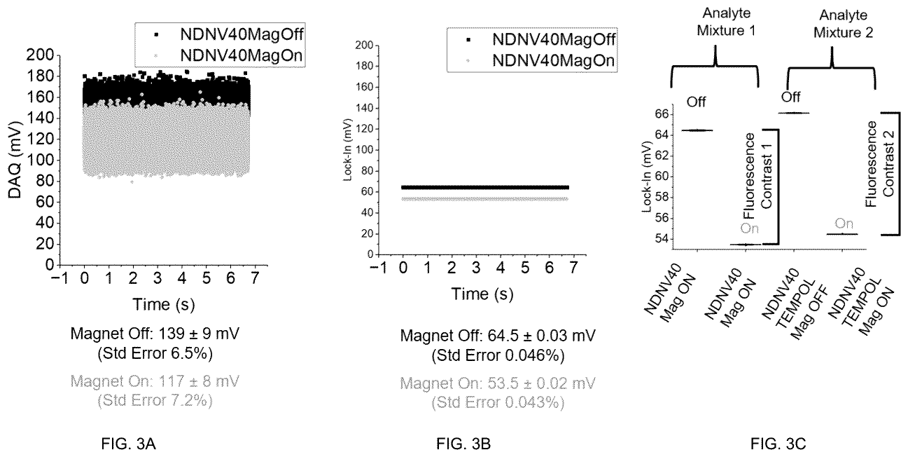

FIGS. 3A, 3B and 3C are illustrations of the reduction in overall noise provided by optical modulation and subsequent phase-dependent detection through lock-in amplifier as exemplified by fluorescence contrast generated by a static magnetic field. In this example, fluorescence data from 40 nm fluorescent nanodiamond dispersed in deionized (DI) water (0.5 mg/ml) is collected in the presence (on) or absence (off) of a static magnetic field generated by an electromagnet, producing a constant field on the sample of at least 50 mT. The data is collected directly by data acquisition device (FIG. 3A) or after light modulation (674 Hz) and subsequent detection via lock-in amplifier (FIG. 3B). The standard error decreases more than about 140× (FIGS. 3A and 3B), with a variability of <0.05%. FIG. 3C illustrates differentiation of analyte mixtures 1 and analyte mixture 2 as shown by different voltage outputs generated in the presence (on) or absence (off) of a static magnetic field, producing respective different fluorescence contrasts 1 and 2 (FIG. 3C) in absence and presence of paramagnetic analyte. Analyte mixtures 1 and 2 are 40 nm NDNV in DI water (0.5 mg/ml) in absence and presence of Tempol (500 uM) analyte, correspondingly.

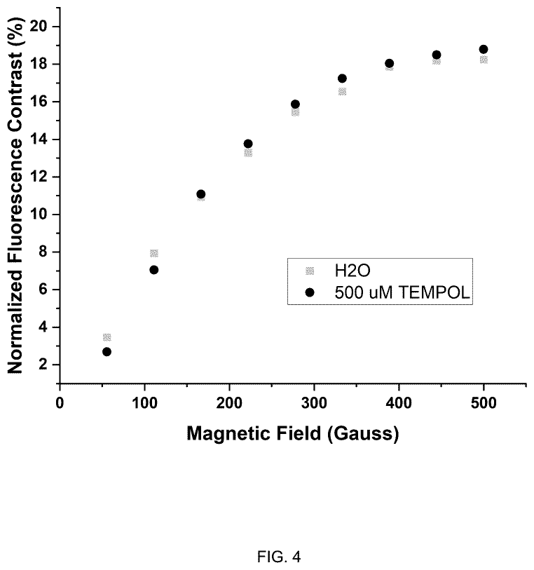

FIG. 4 is an illustration of the effect of strength of external static magnetic field on normalized fluorescence contrast. The measured fluorescence contrast (as for example, in FIG. 3C), was normalized to the signal measured with the magnetic field off. Measurements were done for 40 nm NDNV in DI water (0.5 mg/ml) in absence and presence of paramagnetic analyte (500 uM Tempol). For the given analyte, the normalized fluorescence contrast saturates above approximately 40 mT (400 G), as illustrated in FIG. 4. The dependence on magnetic field is not necessarily the same for each analyte, whereby contrast and normalized contrast values in the presence of analyte may increase or decrease in relation to the contrast measured in the absence of analyte (FIG. 4). Selection of the field strength for experiments can vary from close to zero magnetic fields (for example 1-50 Gauss, or 30-50 Gauss) to larger fields when the dependence of contrast on magnetic field saturates (approximately above 400 Gauss). In the majority of experiments described below, a magnetic field of approximately 300-500 Gauss was used, and up to 1500 Gauss.

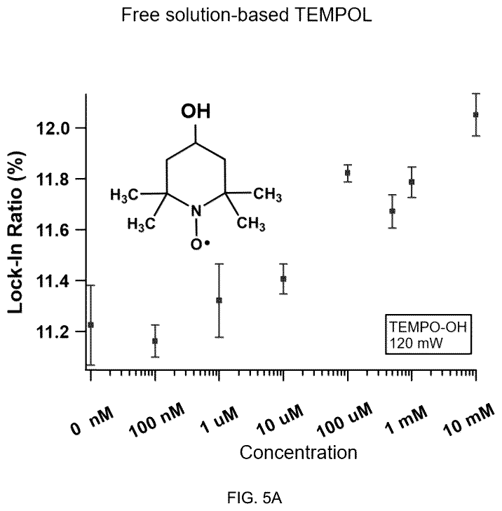

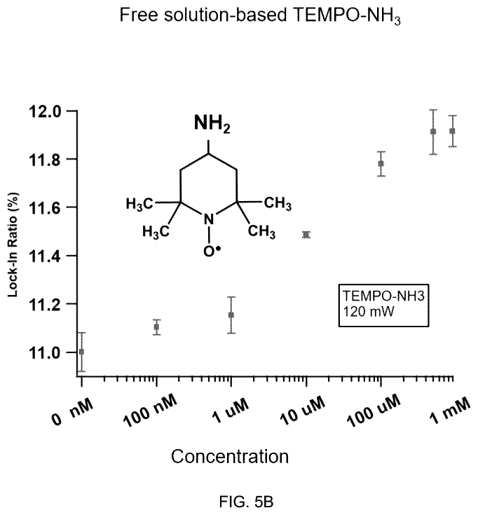

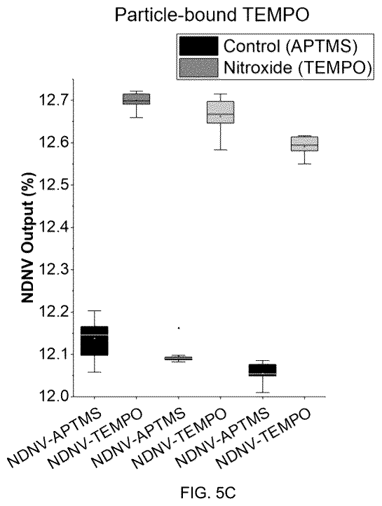

FIGS. 5A, 5B and 5C are illustrations of the measurement of stable radicals, both dispersed free in solution and bound to the particles surface. Chemically stable radicals such as those in the nitroxide of TEMPOL were analyzed. These analytes can be detected by comparing the fluorescence contrasts measured by magnetic modulation of NDNV fluorescence in the presence and absence of analyte. NDNV concentration is similar in mixtures of NDNV and stable radicals at different concentrations (FIGS. 5A and 5B). FIG. 5A illustrates dependence of normalized fluorescence contrast of 40 nm NDNV (0.5 mg/mL) on concentration of free TEMPOL molecules dispersed in aqueous solution. TEMPOL does not affect pH or aggregation state of NDNV. FIG. 5B shows a similar concentration dependence of normalized fluorescence contrast for 40 nm NDNV (0.5 mg/mL) with free TEMPO-NH3 dispersed in aqueous solution. In a different embodiment, FIG. 5C illustrates that 70 nm NDNV with TEMPO molecules bound to the NDNV surface is readily differentiated from non-TEMPO functionalized material. The measurements were repeated three times by alternating the samples with and without paramagnetic analyte to show reproducibility of the results (FIG. 5C).

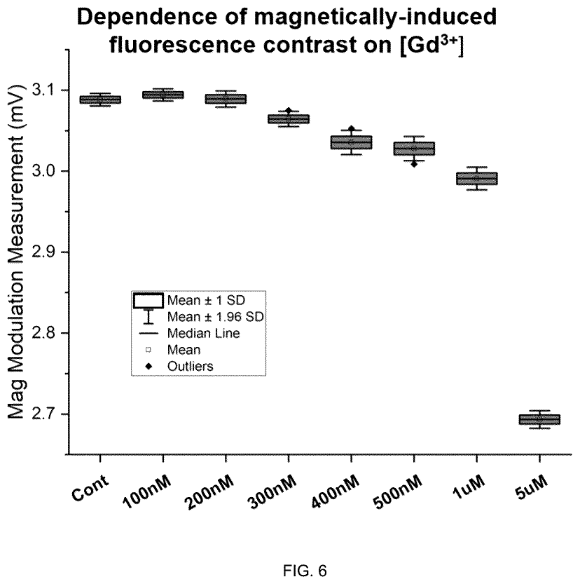

FIG. 6 is an illustration of the fluorescence contrast from magnetic modulation of nanodiamond fluorescence (40 nm NDNV at 0.5 mg/mL) as a function of Gd3+ concentration.

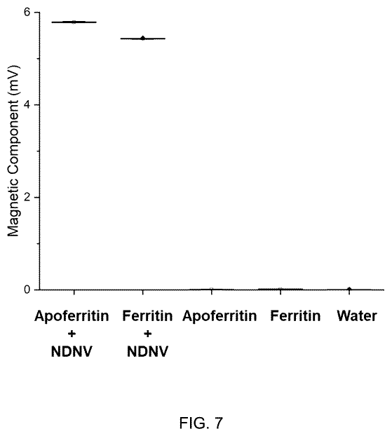

FIG. 7 is an illustration of differentiation of iron-containing ferritin from apoferritin (same protein, but no iron present) by measuring the normalized fluorescence contrast by magnetic modulation of nanodiamond fluorescence for 40 nm NDNV at 0.5 mg/mL dispersed in DI water in a mixture with analyzed proteins. Proteins are measured at equal concentrations 2.5 mg/mL

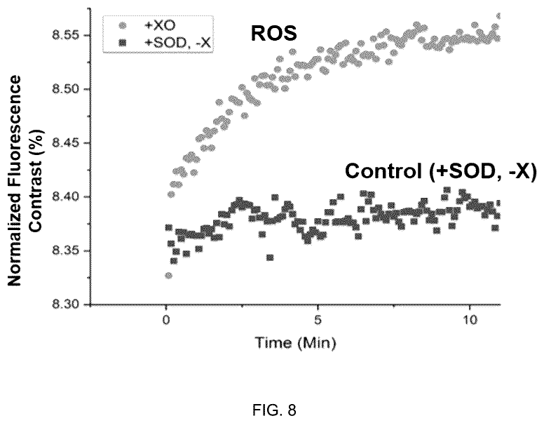

FIG. 8 is an illustration of in vitro detection where the ROS generation by the enzyme xanthine oxidase (XO) was followed over ten minutes. XO is a commonly used model of ROS because in the presence of xanthine (X) ROS are produced including free radicals (e.g. superoxide, hydroxyl radicals) and peroxide. NDNV sensors of 40 nm in size showed clear response related to the metabolic reaction of catalyzing the oxidation of xanthine by XO and producing ROS. When compared to enzyme activity without xanthine and additionally with a scavenger for superoxide (SOD), NDNV did not produce any significant change in signal over time in the control sample.

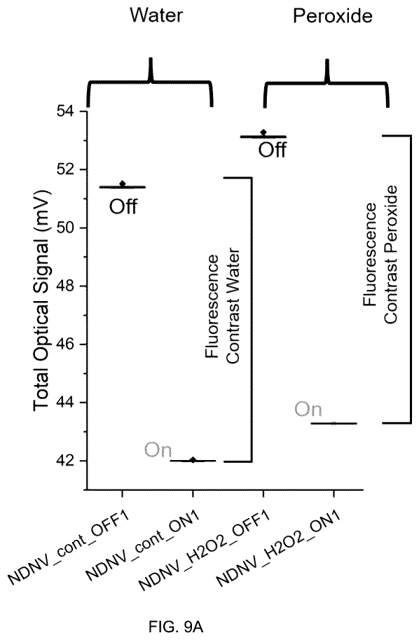

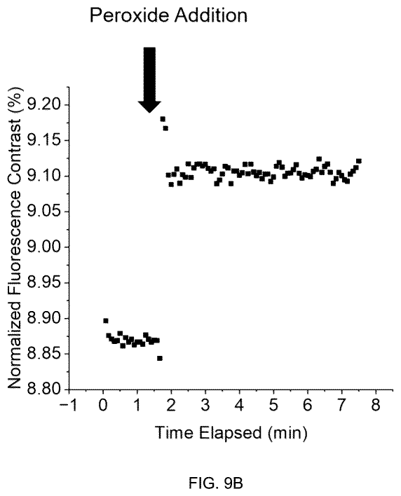

FIGS. 9A and 9B are illustrations of detection of hydrogen peroxide by magnetic modulation of nanodiamond fluorescence (40 nm NDNV at 0.5 mg/mL) by comparing fluorescence contrast in samples where NDNV is dispersed in DI water and in a mixture with hydrogen peroxide. In FIG. 9A, hydrogen peroxide containing sample is readily differentiated from a control sample with water in a static magnet configuration. In FIG. 9B, in a setup for monitoring fluorescence contrast in a sample over time, upon addition of peroxide a significant change in fluorescence contrast is observed. Peroxide concentration is 0.4% in both cases.

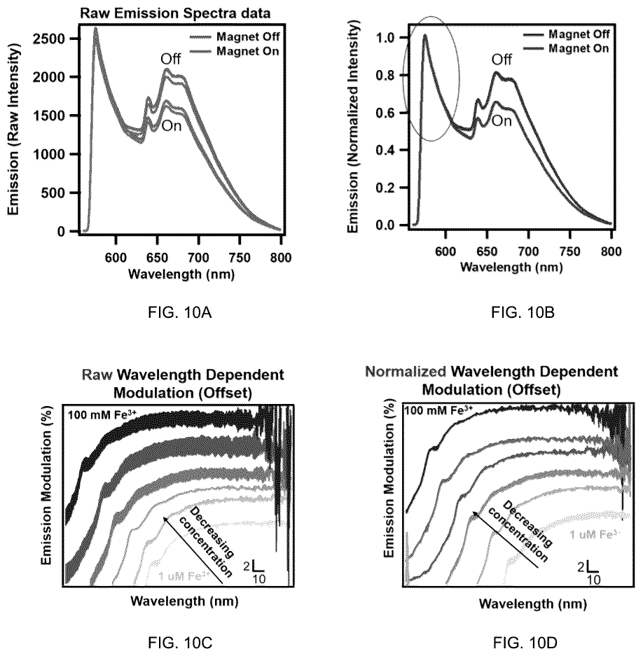

FIGS. 10A, 10B, 10C and 10D are illustrations of sensing of iron (III) ions where normalization of modulated fluorescence intensity of NV centers to unchanged fluorescence of NVN centers in diamond is used to reduce noise. FIG. 10A shows typical spectra variability in repetitive measurement of the same sample due to drift over time between magnet-off and -on states. After normalization of spectra to the NVN center (FIG. 10B), variation in the spectra is dramatically reduced. Utilization of fluorescence intensity normalization to NVN is shown in the measurement of Fe3+ over concentrations of 100 mM to 1 uM. When multiple replicates are run, environmental drift, and/or sample variation can result in noise in the raw wavelength dependent fluorescent contrast (FIG. 10C). After normalization as described by FIG. 10B, the resulting noise in the fluorescence contrast is reduced dramatically (FIG. 10D). This normalization can be applied in any implementation of magnetic modulation with or without optical modulation.

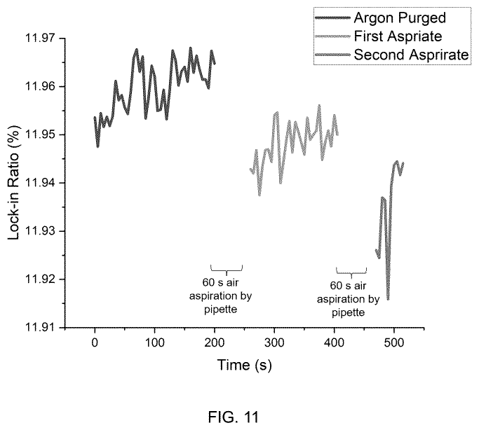

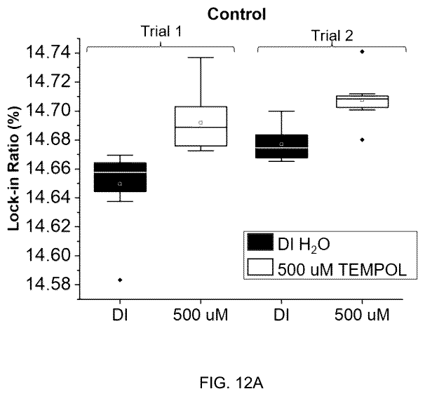

FIG. 11 is an illustration of detection of differences in the amount of oxygen dissolved in DI water using 40 nm fluorescent nanodiamond (1 mg/mL). The normalized fluorescence contrasts are different for samples where oxygen content was first reduced by purging argon through the sample and for two samples where dissolved oxygen concentration was increased by aeration of the argon-purged sample sequentially with two aeration phases.

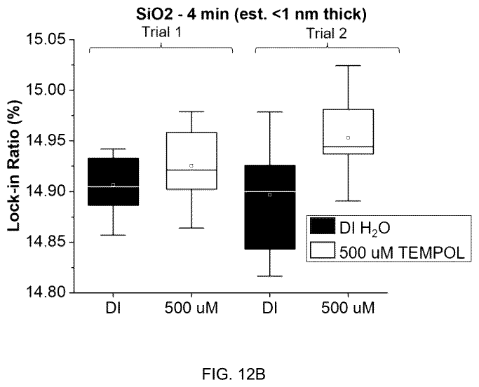

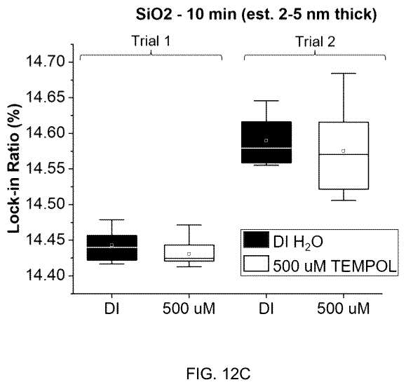

FIGS. 12A, 12B and 12C are illustrations of the effect of SiO2 coating on the sensing capability of 50 nm fluorescent nanodiamond (0.5 mg/mL) in a mixture of 50% ethanol and 50% aqueous solution. FIG. 12A illustrates well discriminated normalized fluorescent contrast in the analyzed mixture for carboxylated NDNV with and without TEMPOL. The ability to discriminate TEMPOL from control was found to decrease as the shell thickness was increased as illustrated in FIG. 12B and FIG. 12C, where silica layers of different thicknesses were produced by terminating the reaction at 4 min and 10 min, correspondingly. Estimated thicknesses of silica shells are about 1 nm and up to about 5 nm for NDNV in FIG. 12B and FIG. 12C, correspondingly.

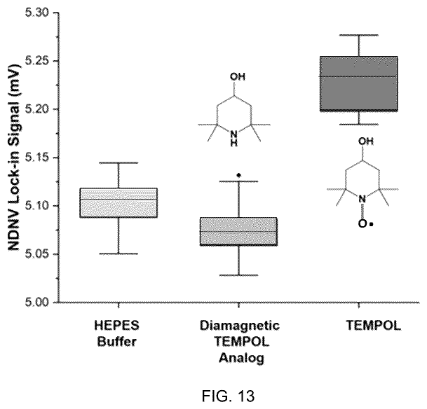

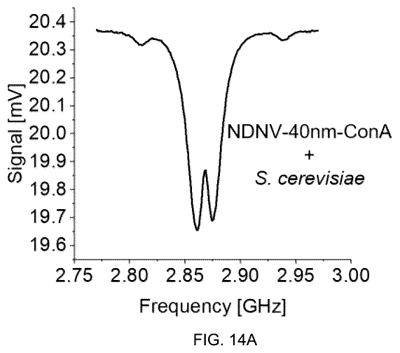

FIG. 13 is an illustration of the effect of differentiation of analytes containing free-radical TEMPOL and its diamagnetic analog using magnetically-induced fluorescence contrast in NDNV. All measurements were performed with 40 nm NDNV (0.5 mg/mL) in 10 mM HEPES buffer. Analyte concentration was set at 500 uM. TEMPOL contains a stable radical whereas the diamagnetic analog (2,2,6,6-tetramethylpiperidin-4-ol) does not. Fluorescence contrasts in samples where NDNV is dispersed in HEPES buffer containing TEMPOL are readily differentiated from a similar mixture containing HEPES buffer alone or the diamagnetic analog of TEMPOL.

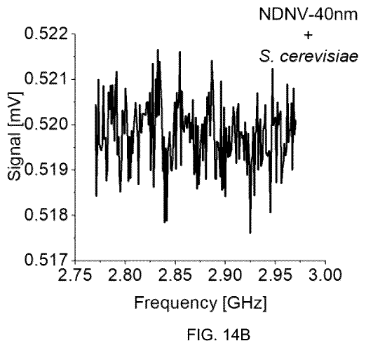

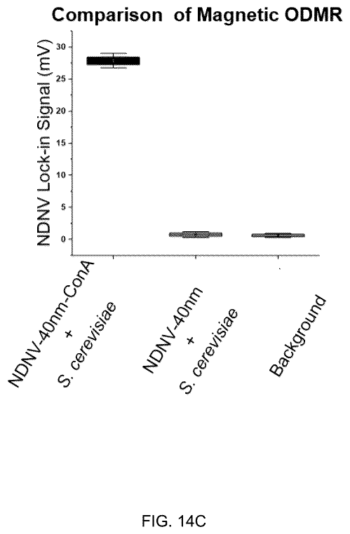

FIGS. 14A, 14B, 14C is an illustration of an analysis of yeast incubated with targeted (NDNV-40 nm-ConA) or untargeted (NDNV-40 nm) nanodiamond by two complementary methods, continuous-wave microwave optically detected magnetic resonance and magnetically-induced fluorescence contrast. In FIG. 14A, ODMR spectrum from NV centers in NDNV-40 nm-ConA incubated with yeast shows detectable NV signal after cells washing. In FIG. 14B, ODMR spectrum of yeast incubated with untargeted NDNV-40 nm show no detectable NV signal after cells washing. FIG. 14C illustrates comparison of magnetically induced fluorescence contrast measured for NDNV-40 nm-ConA incubated with yeast cells showing readily detectable NV signal after the cells washing, while in the yeast sample incubated with the untargeted NDNV the contrast is indistinguishable from background experimental noise.

### EXAMPLES

### Example 1. Noise Reduction by Using Optical Modulation in Combination with a Static (DC) Magnetic Field

In this example, 40 nm fluorescent nanodiamond (NDNV40) containing approximately 1.5 ppm of NV centers were dispersed in distilled water at 0.5 mg/mL and flowed through a sample unit (41 in FIG. 1) at rate of 25 uL/min using a syringe pump. This sample unit was a glass capillary (1 mm ID, 200 um thick wall) cooled by an air blower. Excitation and detection of NDNV fluorescence in a continuous flow of a sample, if a sample is in liquid state, provides averaging of a signal over larger sample volume mitigating possible sample heterogeneity and/or preserving the optical environment and as a result contributes to the noise reduction. Data was collected as configured in FIG. 1 where the optical path was focused into the capillary to deliver excitation light (continuous wave 532 nm laser (Coherent)) to the sample and to direct emission light to the photodetector (51), in this case an avalanche photodiode detector (Thorlabs APD440A). Light reaching the detector passed a longpass 650 nm filter. Two configurations were run. In one configuration, light was not modulated (21, f2=0 Hz) and voltage data from the photodetector was read directly by a data acquisition device 81 (National Instruments USB-6218) as illustrated in FIG. 3A. In a second configuration, the excitation light was modulated by an optical chopper (Thorlabs MC2000B) (21, f2=674 Hz) which also provides the relevant reference signal for processing with lock-in amplifier. The output of photodetector 51 was then read by a lock-in amplifier (SRS 850), the output of which was recorded by the same data acquisition device as in the first configuration. The output signals for the modulated light are illustrated in FIG. 3B. In both configurations, data was collected in the absence of a magnetic field (B=0 mT) or in the presence of a static DC magnetic field (61, f1=0 Hz), generated with a constant current passed through an electromagnet (Uxcell) creating a field of approximately 50 mT. The result of these two configurations was a decrease in standard error by more than 140× by use of the optical modulation in measurement of fluorescence modulation contrast produced by a static magnetic field. The variability of the signals with and without magnetic field applied using optical modulation was dramatically decreased to <0.05% (FIG. 3B). In the context of a chemical species detection, the configuration related to FIG. 3B was modified to compare two analyte mixtures: analyte mixture 1 consisted of 40 nm NDNV (0.5 mg/ml) in DI water while analyte mixture 2 consisted of 40 nm NDNV (0.5 mg/ml) in the presence of 500 uM TEMPOL (FIG. 3C). Comparison of the states with magnetic field-on and -off generated specific contrast values, respectively fluorescence contrast 1 (17.07%) and fluorescence contrast 2 (17.66%), allowing for differentiation of the analyte.

The dependence of magnetically modulated fluorescence contrast on magnetic field strength was also investigated. FIG. 4. is an illustration of the effect of external magnetic field on normalized fluorescence contrast. For a given analyte (500 uM TEMPOL) mixed with 40 nm NDNV (0.5 mg/ml), contrast saturates above approximately 40 mT (400 G). The relation to magnetic field is not necessarily the same for each analyte, whereby contrast values may increase or decrease in relation to a chemical species dependent on field strength.

### Example 2. Detection of Stable Radicals in Solution by Analyzing Magnetically Modulated Fluorescence Contrast of Nanodiamonds

In this example, the specified analyzed mixtures flowed through a sample unit at a rate of 25 uL/min as described in Example 1. A time-varying (AC) magnetic field (61, f1=41 Hz) was generated with an electromagnet (Uxcell). The electromagnet was driven by a current supply modulated by transistor-to-transistor logic (TTL) creating a field of approximately 50 mT at the sample unit. The TTL reference was generated from the lock-in amplifier. The output of photodetector 51 was then split and read by two separate lock-in amplifiers (SRS 850 and SRS 830) simultaneously, to process optical modulation and magnetic modulation at related frequencies, correspondingly.

The output of each lock-in amplifier was read and averaged by a data acquisition device 81 (National Instruments USB-6218). Normalized fluorescent contrast was generated by the ratio of the lock-in outputs (magnetic modulation output divided to optical modulation output). The concentration dependence of two chemical species, specifically the stable radicals TEMPOL and TEMPOL-NH3, were then studied (FIGS. 5A and 5B). Radicals such as those in the nitroxide of TEMPOL are chemically stable over time. In each case, the sample capillary was washed with a flow of DI water between flows of analyte mixture with fluorescent nanodiamonds, where the concentration of nitroxides varied from 0 to 10 mM. Experiments were done with 40 nm nanodiamonds containing 1.5 ppm of NV centers (NDNV) at 0.5 mg/mL particle concentrations. For both analytes, no aggregation of diamond is observed in these concentrations and the effect of pH is minimal. For both analytes, fluorescence contrast strongly corelated with analyte concentration.

### Example 3. Detection of Stable Radicals Conjugated to the Particle Surface by Analyzing Magnetically Modulated Fluorescence Contrast of Nanodiamonds

In this example, measurements were taken as described in example 2. In this case, the analyzed mixture consisted of 70 nm fluorescent nanodiamond with approximately 3 ppm of NV centers. The NDNV were either coated with TEMPOL covalently linked to the surface through (3-aminoproply)trimethoxysilane (APTMS) or functionalized with only APTMS without TEMPOL. Samples were alternated in the flow capillary over the course of 30 minutes with washing the capillary by DI water between alternated samples. The samples with TEMPOL were readily differentiable from unlabeled samples as illustrated in FIG. 5C from comparison of normalized fluorescence contrasts of samples with and without paramagnetic analyte.

### Example 4. Detection of Gd3+ Ions in Solution by Analyzing Magnetically Modulated Fluorescence Contrast of Nanodiamonds

In this example, measurements were taken as in example 2, with the modification that for this analysis the direct fluorescence contrast was taken from the lock-in amplifier measuring magnetic modulation, without normalization to optically modulated output. The diamond concentration was kept constant for each measurement. The analyzed samples consisted of mixtures of 40 nm fluorescent nanodiamond (0.5 mg/mL) with 1.5 ppm NV and gadolinium nitrate at concentrations from 5 uM to 0 uM. As shown in FIG. 6, measured fluorescence contrast strongly correlates to the gadolinium concentration at approximately >300 nM of Gd3+ concentration.

### Example 5. Differentiation of Apoferritin and Ferritin by Analyzing Magnetically Modulated Fluorescence Contrast of Nanodiamonds

In this example, measurements were taken as in example 4, utilizing direct measurement of magnetically modulated fluorescence contrast with constant diamond concentration in the analyzed samples. Ferritin is a spherical iron-containing protein that plays a major role in iron homeostasis in the animal and human body. Apoferritin is the iron-free version of this protein that is molecularly identical but does not contain iron. Solutions of ferritin or apoferritin in DI water were made at equal mass concentration (2.5 mg/mL) with 40 nm fluorescent nanodiamond containing 1.5 ppm NV at 0.5 mg/mL. Solutions of proteins without nanodiamond (at concentration of proteins 2.5 mg/mL), and additional water control were also analyzed. As shown in FIG. 7, ferritin and apoferritin solutions without NDNV produced no signal and were not differentiable from water. With fluorescent nanodiamond present as a sensor, ferritin was readily differentiable from apoferritin showing a reduction in magnetically modulated fluorescence contrast. Such measurements are relevant for diagnostics of iron overload and iron deficit disorders by analyzing iron content in ferritin in blood or in processed blood using NDNV sensors.

### Example 6. In Vitro Measurement of the Metabolism of the Enzyme Xanthine Oxidase

Xanthine oxidase (XO) is an enzyme which catabolizes, among other compounds, xanthine (X) into uric acid, consuming oxygen and producing reactive oxygen species such as superoxide and hydrogen peroxide. The progress of xanthine oxidation over time as compared to a control were measured by monitoring changes in normalized fluorescence contrast, in a manner as described in example 2. Data was collected in real time over the course of 10 minutes. Components of the reaction included the enzyme XO and its substrate (X). Pentetic acid (DTPA) chelates metals to prevent enzyme deactivation. Superoxide dismutase (SOD) was used as a control to prevent superoxide formation. DEPMPO is a diamagnetic molecule that can react with radicals to form a stable, paramagnetic molecule. For both the xanthine oxidase sample and control, all components were added except XO. The reaction composition consisted of 25 uM pentetic acid (DTPA), 150 uM X, 7 mM DEPMPO spin trap, 24 mU/mL XO, and 0.5 mg/mL 40 nm fluorescent nanodiamond (1.5 ppm NV). The control reaction consisted of 25 uM DTPA, 300 U/mL SOD, 7 mM DEPMPO spin trap, 24 mU/mL XO, and 0.5 mg/mL 40 nm fluorescent nanodiamond (1.5 ppm NV). XO was added last and immediately before the measurement began to initiate oxidation. The control sample with SOD and no substrate X produced no significant change in normalized fluorescence contrast over the 10 min observation period (FIG. 8). By comparison, the active XO solution showed a highly differentiable change in normalized fluorescence contrast over time (FIG. 8).

### Example 7. Measurement of Hydrogen Peroxide by Analyzing Magnetically Modulated Fluorescence Contrast of Nanodiamonds

Peroxide is a reactive species that can both generate ROS in solution as well as react with the surface of fluorescent nanodiamond, though peroxides themselves are not paramagnetic species. Hydrogen peroxide plays an important role in oxygen-involving metabolism in biological systems, may regulate a wide variety of biological processes and is produced internally and externally to cells.

In this example, magnetically modulated fluorescence contrast was measured in two configurations: in a static magnetic configuration, with a setup similar to example 1 (FIG. 9A) and in a setup using an oscillating magnetic field similar to example 2 (FIG. 9B). In the static configuration, the fluorescence of 40 nm fluorescent nanodiamond (1.5 ppm NV) at 0.5 mg/ml was measured without a magnetic or with a DC magnetic field of approximately 50 mT. Measurements were done with water and with 0.4% hydrogen peroxide. Comparison of the on/off magnetic states provide respective fluorescence contrast for water (18.30%) and fluorescence contrast for peroxide (18.57%) (FIG. 9A). Similarly, data was collected with an oscillating magnetic field (50 mT, 41 Hz) where 40 nm fluorescent nanodiamond (1.5 ppm NV) at 0.5 mg/ml was measured for 2 minutes, after which concentrated hydrogen peroxide was added to the particle reservoir, causing a significant increase in the normalized fluorescence contrast directly correlated to the peroxide addition (FIG. 9B).

### Example 8. Reduction of Noise in Magnetically Modulated Fluorescence Contrast Measurements Due to Normalization to Unchanged Fluorescence Intensity of NVN Centers

As opposed to NV centers, fluorescence in diamond from NVN centers (H3 centers) is invariant to modulation by external fields. In this example, solutions of fluorescent nanodiamond containing a mixture of NV and NVN centers within individual particles were produced by high temperature annealing (1600° C.) of electron-irradiated (5E18 e/cm² dose) 1 um nanodiamonds. Suspension in DI water of the fluorescent nanodiamond particles was dropped into a well-plate format and dried. Measurements were collected on an inverted fluorescence microscope with mercury arc lamp illumination passed through a 517/20 nm bandpass filter for excitation and emission light collected via 561 nm longpass filter. Data was collected on a spectrometer with a CCD detector (Ocean Optics HR2000). A static DC magnetic field was generated by a rare-earth neodymium magnetic (K&Js Magnets) placed over the well containing the sample to generate a field greater than approximately 150 mT with covering to prevent light reflections. When the field was applied to the sample, changes in the spectral intensity occur in the region of the negatively charged NV center, predominantly >570 nm. Because of variability in the light source intensity, replicate measurements of the same sample showed variability in fluorescence (FIG. 10A). Normalization of the spectra to the NVN center intensity (here at approximately 573 nm based on the cut-off of the filter) removes the variability in measurements for magnetic-On or magnet-Off states (FIG. 10B). The utility of this is apparent in wavelength-dependent magnetically modulated fluorescence contrast measurements, where at each wavelength contrast is plotted as: 100×(fluorescence without magnet−fluorescence with)/fluorescence without magnet. Measurements were performed with iron(III) nitrate, dried in well plates with diamond containing NV and NVN centers, with varying concentration of 1 uM to 100 mM. In this format, before normalization none of the samples were differentiable, while after normalization detection was realized down to 100 uM, without optical modulation or phase-dependent magnetic detection to reduce noise.

### Example 9. Measurement of Oxygenation of Water by Analyzing Magnetically Modulated Fluorescence Contrast

Oxygen is a paramagnetic molecule present in solution typically at equilibrium with the environment in solution. Oxygen content can change due to changes in the ambient environment or biological processes producing or consuming oxygen in solution, such as the oxidation of metabolites or the generation of ROS. To measure changes in dissolved oxygen content, 40 nm fluorescent nanodiamond with 1.5 ppm NV at 1 mg/ml were put into an amber glass vial with septum. The septum was pierced with a needle placed under the surface of the liquid near the bottom and an additional needle added as a vent. Argon was bubbled through the solution for 45 minutes. To transport for measurement, the vent needle was removed to generate positive pressure, and the vial was then sealed with parafilm after removing the argon line.

Samples were then analyzed in a manner similar to example 2 for measuring fluorescent contrast. The argon-purged nanodiamond sample was drawn directly from the vial as soon as the vial was opened. After baseline data was collected, some of the solution was moved to a microfuge tube and the sample was vigorously aspirated with a micropipetter for one minute. After measuring the aspirated sample, this process was repeated with further aspiration. Upon each aspiration event, where oxygen was added to the sample, the measured normalized fluorescence contrast was lowered.

### Example 10. Effect of an SiO2 Layer on Magnetically Modulated Fluorescence Contrast of Nanodiamonds and Measurement of a Chemical Species

The surface of fluorescent nanodiamonds can play the role of the ability for NV to respond to their environment, causing changing in T1 relaxation times of NV and impacting the magnetically modulated fluorescence contrast value. Samples of 50 nm fluorescent nanodiamond (approximately 1 ppm NV) were coated with layers of SiO2, including samples without silica layer, and layers produced by terminating the reaction at different time points. Layers were created by a variant of the Strober process, where layers were formed by base-catalyzed condensation of tetraethylorthosilicate onto the particle surface in a time dependent fashion. The coated (or control uncoated) samples in ethanol were then mixed with either DI water or 1 mM TEMPOL in water producing solutions of 0.5 mg/mL fluorescent nanodiamond with 500 uM TEMPOL in water or pure water (control), with a final solution mixture of 1:1 water and ethanol. The ability to discriminate the external chemical species such as TEMPOL from control was found to decrease as the shell thickness was increased (FIG. 12).

### Example 11. Differentiation of Molecules Containing Stable Radicals and their Diamagnetic Analog by Analyzing Magnetically Modulated Fluorescence Contrast of Nanodiamonds in the Analyzed Mixture

In this example, measurements were taken as described in example 2. All measurements were performed with 40 nm NDNV (0.5 mg/mL) in 10 mM HEPES buffer. Analyte concentration was set at 500 uM. TEMPOL contains a stable radical whereas the diamagnetic analog (2,2,6,6-tetramethylpiperidin-4-ol) does not. As illustrated in FIG. 13, fluorescence contrasts in samples where NDNV is dispersed in HEPES buffer with TEMPOL are readily differentiated from the mixture containing its diamagnetic analog in HEPES buffer and a solution with NDNV only in HEPES buffer.

### Example 12. Functionalization of NDNV for Colocalization with Cell Membrane

Reactive oxygen species (ROS) play an important role in cell cultures in bioreactors including, but not limited to cultures of mammalian cells, stem cells, bacteria and yeast. For example, ROS play a major role in yeast's metabolism and relate to the yield of desired fermentation end products either directly through the anabolic pathways themselves or indirectly through oxidative stress which reduces product output. In order to use NDNV as sensors of ROS in cell cultures, NDNV have to be associated with cells, being distributed internally or externally to cells and in certain embodiments, to be targeted to specific sites (for example, mitochondria) within cells or externally to cells. For intercellular delivery, NDNV can be internalized by cells directly (e.g. by mammalian cells), delivered using electroporation, ballistic approaches (e.g. a gene gun) or other methods known in the field. NDNV can be also associated with cell membranes for measurement of extracellular ROS production, where ROS can be produced, for example, through the activity of nicotinamide adenine dinucleotide phosphate (NADPH) oxidase, or diffused through a cell membrane from intercellular region (e.g. hydrogen peroxide).

This example demonstrates how NDNV can be colocalized with the yeast cell membrane in order to be used for further detection of analyte in the vicinity of cell membrane externally to a cell. For this, applicant functionalized NDNV with concanavalin A (ConA), a carbohydrate binding protein which binds especially well to α-D-mannosyl and α-D-glucosyl on cell membrane. This lectin was chosen as a robust protein which is readily available. For labeling of 120 nm NDNV with ConA (NDNV-120 nm-ConA), functionalization was done via EDC/NHS mediated carbodiimide coupling, followed by sequential centrifugal washing steps. Yeast (S. cerevisiae) cells were co-incubated with commercially available fluorescein isothiocyanate (FITC) modified concanavalin A (FITC-ConA) and NDNV-120 nm-ConA and washed from the unbound labels. Labeling was done in the presence of 0.1% bovine serum albumin as a blocking agent to ensure specific labeling. The cells were imaged using confocal microscope (Zeiss LSM 880) and 561 nm laser excitation and 570-677 nm emission to visualize NDNV and 488 nm laser excitation and 490-561 nm emission to visualize FITC. Fluorescence microscopy revealed unambiguous association of NDNV120 nm-ConA with the yeast surface, correlating to the standard FITC fluorophore.

### Example 13. Preparation of NDNV for Detection of Analyte in Yeast Cells Culture

For sensing of metabolites, size of the NDNV particle sensors becomes an important factor. Smaller particles (less than about 70 nm) are more effective sensors because a larger number of NV centers are susceptible to adjacent analytes due to larger surface area per unit mass. However, smaller particles have fewer NV centers per particle making their fluorescent output lower. In this example, NDNV particles with about 40 nm size (NDNV-40 nm) containing about 1 ppm of negatively charged NV centers was used in experiments. To functionalize this size regime with ConA, our functionalization methods were altered in comparison with example 12 to reduce aggregation. Functionalization still occurs via EDC/NHS mediated carbodiimide coupling, however as opposed to reaction of 120 nm particles which involved sequential centrifugal washing steps, here all modifications occurred directly in suspension, with selective activation and quenching steps, providing functionalized NDNV-40 nm-ConA. The result was a lower degree of interparticle aggregation and/or bridging of particles by the functionalization protein. One group of yeast cells (S. cerevisiae) was co-incubated with NDNV40 nm-ConA and washed from unbound NDNV40 nm-ConA. Another, a control group was incubated with NDNV-40 nm (untargeted) and washed. Two cell groups were analyzed in fluorescent inverted microscope (Olympus IX71) using 10× objective. During imaging using 532 nm wavelength for excitation of NV fluorescence, it was noticed that the background autofluorescence of yeast is significant, such that NDNV cannot readily be differentiated from the background via conventional microscopy alone. Analysis of yeast incubated with targeted (NDNV-40 nm-ConA) or untargeted (NDNV-40 nm) nanodiamond to detect presence of NDNV based on spin properties of NV centers was done by two complementary methods of analysis, continuous-wave microwave optically detected magnetic resonance (CW ODMR) or magnetically-induced fluorescence contrast. The essential components of the setup for measurement of ODMR spectra in yeast cells incubated with NDNV comprised an inverted microscope for fluorescence imaging, a light source, a photodetector, an arbitrary wave generator (AWG), a signal generator, a power amplifier, microwave antenna, an oscilloscope, and a PC equipped with remote control software. Measurements of magnetically-induced fluorescence contrast in yeast cells incubated with NDNV were done using the setup illustrated in FIG. 1. Both methods were able to clearly resolve the two samples incubated with NDNV when NDNV was functionalized with ConA for targeting of the cell membrane or using untargeted NDNV. FIG. 14A and FIG. 14B illustrate comparison of ODMR spectra from targeting NDNV-40 nm-ConA in the presence of yeast analytes against a sample incubated with untargeted NDNV-40 nm. The targeting sample (FIG. 14A) shows a clear ODMR signature, while in the untargeted sample (FIG. 14B) the level of diamond present, potentially due to non-specific binding, is low, so that an ODMR spectrum cannot be generated. Similarly, comparison of magnetically induced fluorescence contrast illustrated in FIG. 14C, where the sample of yeast cells incubated with NDNV-40 nm-ConA shows readily detectable diamond signal (fluorescence contrast) as compared to the untargeted sample, which is indistinguishable from background experimental noise. Measurement of NDNV ODMR and magnetically induced fluorescence contrast in the presence of highly autofluorescent yeast incubated with targeted 40 nm NDNV represents a critical step towards the adaption of our analyte sensing protocols in situ. Importantly, labeling was tested with R. toruloides (wild type), confirming that our targeting strategy is useful for different yeast strains. Being able to detect the ODMR spectrum and fluorescence contrast provides feasibility for detection of ROS analyte by measuring changes in the ODMR spectra or in the fluorescence contrast upon activation of production of ROS, for example, in an enzymatic reaction. Capability of such measurement was illustrated in example 6, illustrating the progress of ROS generation over time in an enzymatic reaction using xanthine oxidation and as compared to a control by monitoring changes in normalized magnetically induced fluorescence contrast. NDNV targeted cell membranes can be used specifically for measurement of extracellular ROS produced, for example, through the activation of NADPH oxidase, a membrane-bound enzyme complex facing the extracellular space. In one embodiment, membrane-bound NADPH oxidase in yeast cells can be activated externally, and ROS generation can be detected by membrane-bound NDNV directly (as in example 6) or by using diamagnetic spin traps reacting with the produced ROS and generating stable free radicals which then can be detected by the NDNV.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A method of monitoring of the fluorescence intensity of a diamond particle comprising:
a. taking plurality of diamond particles capable of exhibiting fluorescence in spectral ranges of about 640 nm to about 800 nm;
b. exciting fluorescence of the particles by optical radiation, and determining fluorescence intensity;
c. applying a magnetic field and optical radiation to the particles and determining fluorescence intensity; and
d. comparing the fluorescence intensity of b) with the fluorescence intensity of c).

2. The method according to claim 1, where a) further comprises introducing diamond particles to a sample that may contain a chemical species to be analyzed and forming an analyzed mixture.

3. The method according to claim 1, where d) further comprises providing a fluorescence contrast; and where using the fluorescence contrast comprises determining the presence of paramagnetic chemical species.

4. The method according to claim 1, where applying a magnetic field to the particles further comprises modulating the fluorescence intensity by the applied magnetic field.

5. The method according to claim 1, further comprising fluorescent diamond particles containing plurality of NV and H3 color centers, and where fluorescence intensity of NV centers measured with and without applying magnetic field is calibrated to the unchanged fluorescence intensity produced by excitation of H3 centers.

6. The method according to claim 1, where the plurality of the fluorescent diamond particles is further combined with a cell, organism, organ, tissue, fluid, matrix or a substrate, where the substrate is electronic component, a tag, a tracer, a label, a polymer, and an optically transparent solid.

7. A method of analyzing a paramagnetic chemical species comprising:
a. introducing diamond particles capable of exhibiting fluorescence containing at least a single NV center to a sample that may contain a paramagnetic chemical species to be analyzed and forming an analyzed mixture;
b. first applying optical radiation to the analyzed mixture, and detecting the resulting fluorescence intensity using a detector;
c. second, after the fluorescence intensity is detected, applying optical radiation, and applying a magnetic field to the analyzed mixture and detecting the resulting fluorescence intensity using a detector;
d. comparing detected fluorescence intensity in (b) and (c), to provide a fluorescence contrast; and
e. using the fluorescence contrast, determining the presence of paramagnetic chemical species.

8. The method according to claim 7, where concentration of the fluorescent diamond particles in the analyzed mixture is known.

9. The method according to claim 8, where presence of paramagnetic chemical species is defined by comparison between values of a fluorescence contrast in the analyzed mixture and a control sample, where the paramagnetic chemical species are absent.

10. The method according to claim 8, where concentration of paramagnetic chemical species in the analyzed mixture is defined from a calibration curve obtained from samples with known concentration of the paramagnetic chemical species.

11. The method according to claim 7, where the fluorescence contrast is normalized by the value of fluorescence intensity detected in b) or c), providing a normalized fluorescence contrast.

12. The method according to claim 11, where presence of paramagnetic chemical species is defined by comparison of values of a normalized fluorescence contrast in the analyzed mixture and a control mixture where the paramagnetic chemical species are absent.

13. The method according to claim 7, where concentration of paramagnetic chemical species changes over time and where presence of paramagnetic chemical species is defined from changes in the fluorescence contrast over time.

14. The method according to claim 7, where paramagnetic chemical species comprise including but not limited to: species, structures and molecules with unpaired electrons; free radicals, paramagnetic metal ions, paramagnetic metal ion chelates, reactive oxygen species, reactive nitrogen species, reactive sulfur species, ferritin, metalloprotein, oxygen molecules, gaseous molecules containing unpaired electrons, gaseous molecules dissolved in fluid, spin traps, chemical traps; free radicals in proteins, antibodies, antigens; free radicals in nucleic acids; products of enzymatic reactions, products of metabolic reactions.

15. The method according to claim 7, where applying optical radiation in b) and c) comprises at least one of: continuous radiation, optical radiation with periodically time-varying intensity.

16. The method according to claim 7, further comprising modulation of the fluorescent light intensity using an optical chopper and further processing using phase sensitive detection.

17. A method of detection of a chemical species comprising:
a. introducing diamond particles capable of exhibiting fluorescence containing at least a single NV center to a sample that may contain the chemical species to be analyzed and forming an analyzed mixture;
b. first applying optical radiation with periodically time-varying intensity to the analyzed mixture using a light source, and detecting the resulting time-varying fluorescence intensity using a detector;
c. second, after the first fluorescence intensity is detected, applying optical radiation with periodically time-varying intensity and a magnetic field to the analyzed mixture and detecting the resulting time-varying fluorescence intensity using a detector,
d. processing the detected fluorescence intensity in (b) and (c) using a phase-sensitive detection and comparing detected fluorescence intensity in (b) and (c), providing fluorescence contrast; and
e. determining the presence of the chemical species from the fluorescent contrast.

18. A method of detection of a chemical species comprising:
a. introducing diamond particles capable of exhibiting fluorescence containing at least a single NV center to a sample that may contain the chemical species to be analyzed and forming an analyzed mixture;
b. applying optical radiation with periodically time-varying intensity and also applying a magnetic field with periodically time-varying magnitude to the analyzed mixture with a magnetic modulation frequency selected from about 1 Hz to about 10,000 Hz, and detecting the resulting time-varying fluorescence intensity using a detector;
c. processing the detected time-varying fluorescence intensity in (b) using a phase-sensitive detection, providing magnetically modulated fluorescence contrast; and
d. using the fluorescence contrast, determining the presence of the chemical species.

19. The method according to claims 7, 17, or 18, where the optical radiation has wavelength between approximately 480 nm and 1100 nm, preferably between 530 nm and 640 nm, and most preferably between 530 nm and 580 nm.

20. The method according to claims 17 or 18, where chemical species comprise: species, structures and molecules with unpaired electrons; free radicals, hydrogen peroxide, peroxides, paramagnetic metal ions, paramagnetic metal ion chelates, reactive oxygen species, reactive nitrogen species, reactive sulfur species, ferritin, metalloprotein, oxygen molecules, gaseous molecules containing unpaired electrons, gaseous molecules dissolved in fluid, spin traps, chemical traps; free radicals in proteins, antibodies, antigens; free radicals in nucleic acids; products of enzymatic reactions, products of metabolic reactions; free radicals in polymers.

21. The method according to claims 7, 17 or 18, where the analyzed mixture further comprises: whole blood, blood plasma, serum, body fluids, fluids, nutritious substance, waste-water, environmental liquids, buffer, cellular membranes, intracellular membranes, cell compartments, organelles, cytoplasm, animal cell, stem cell, eukaryotic cell, prokaryotic cell, cell culture, intracellular fluid, organism, organ, tissue, plant fluids, plant tissue, plant cell, microorganism, bacteria, yeast, yeast membrane, yeast cytoplasm, intercellular fluid in a bioreactor, animal cell in a bioreactor, biomass in a bioreactor, products of fermentation, fermentation biomass, marine bacteria, phytoplankton, seaweeds, corals, microfluidic chip, organ-on-a chip, polymers, plastics, nitrocellulose membrane, optical fiber, matrix or a substrate, where the substrate is electronic component, a tag, a tracer, a label, a polymer, and an optically transparent solid.

22. The method according to claims 7, 17 or 18, where the analyzed mixture is produced by comprising one of: pouring, mixing, shaking, vortexing, incorporation by sonication, ballistic delivery of fluorescent nanodiamonds, ballistic delivery using a gene gun, drying of a mixed suspension, exposing a sample to fluorescent nanodiamond immobilized on a substrate, capillary, wall of a well, optical fiber or inside an optical fiber.

23. The method according to claims 7, 17 or 18, where applying optical radiation with periodically time-varying intensity to the analyzed mixture comprises a light source including a laser in combination with a light chopper, a pulsed laser, a light emitting diode (LED) in combination with a light chopper, a pulsed LED, mercury-arc lamp in combination with a light chopper, or any device and structures known in the art which can provide optical radiation with periodically time-varying intensity.

24. The method according to claim 23, where the periodically time-varying light intensity is further characterized by frequency, amplitude, phase, and waveform.

25. The method according to claim 23, further comprising phase-sensitive measurement of the detected fluorescence signals including but not limited to a lock-in amplifier.

26. The method according to claims 7 or 17, where applying a magnetic field to the analyzed mixture comprises applying a periodically time-varying magnetic field with a magnetic modulation frequency selected from about 1 Hz to about 10,000 Hz, and further comprising phase-sensitive measurement of the detected fluorescence signals.

27. The method according to claim 26 wherein phase-sensitive measurement is done with a lock-in-amplifier.

28. The method according to claims 7 or 17, where applying a magnetic field to the analyzed mixture comprises applying a periodically time-varying magnetic field further characterized by frequency, amplitude, phase, and waveform.

29. The method according to claims 7, 17 or 18, where applying a magnetic field comprises at least one of: electromagnet, solenoid, mechanically moved static magnet, mechanically moved magnetic shielding.

30. The method according to claims 7, 17 or 18, where applying a magnetic field comprises magnetic field strength between approximately 1 Gauss and 5,000 Gauss and most preferably between approximately 400 and 1000 gauss.

31. The method according to claims 7, 17 or 18 comprising applying optical radiation with periodically time-varying intensity providing optical modulation frequency selected from about 10 Hz to about 10,000 Hz and applying a magnetic field with periodically time-varying magnitude providing magnetic modulation frequency selected from about 1 Hz to about 10,000 Hz and further comprises phase-sensitive measurement of the detected fluorescence signal modulated at the optical modulation and magnetic modulation frequencies, where the detected fluorescence signal is processed by at least one of: a dual-frequency lock-in amplifier, two individual lock-in amplifiers, digital lock-in, computational lock-in, and Fourier analysis.

32. The method according to claim 31, further comprising parallel processing of the signals from two lock-in amplifiers synchronized to the same oscillator.

33. The method according to claims 7, 17 or 18, where the detector for the resulting fluorescence intensity is one of including, but not limited to: avalanche photodiode detector, single photon detector, charged coupled device, photomultiplier tube.

34. The method according to claim 32, where the detected resulting fluorescence intensity may be further analyzed by an analog-to-digital convertor.

35. A method according to claim 34 where in the converter is selected from the group comprising of oscilloscope, data acquisition device, and lock-in amplifier.

36. The method according to claims 7, 17 or 18, wherein diamond particles comprise particles with modified surface providing change in the fluorescence contrast compared to particles with unmodified surface comprising addition or removal of surface groups, surface spins, surface dangling bonds, surface charges, dipole-dipole interactions, addition of shells, aluminum shell, silica groups or silica shells.

37. The method according to claim 36, further comprising functionalizing the diamond particles with at least one functional surface group selected from the group consisting of carboxylic, hydroxyl, amino, hydrogen, epoxy, poly(ethylene glycol), poly(glycerol), hydrocarbon chain, hydrocarbon, aromatic, nucleophile, thiol, sulfur, acid, base, silane, aluminum, halogen and fluoro-containing.

38. The method according to claim 36, where the modified surface comprises changes in at least one of surface spins, surface dangling bonds and surface charges due to the presence of analyte paramagnetic chemical species and where the change in the spin properties of the modified surface impacts spin properties of the NV centers and resulting fluorescence contrast.

39. The method according to claim 38, where the changes in surface spins and surface charges of the modified surface cause changes in T1 relaxation time of NV centers resulting in a change in magnetically modulated fluorescence contrast.

40. The method according to claims 7, 17 or 18, where diamond particles are modified with chemical groups which convert transient analyte into stable forms.

41. The method according to claims 7, 17 or 18, where the analyzed mixture is altered to adjust the fluorescence contrast comprising at least one from the group comprising addition of non-analyte calibrants, chemical species, oxygenation, deoxygenation, diluted gaseous species, pH altering species, and pH stabilizing species, salts.

42. The method according to claims 7, 17 or 18, further comprising: conjugating with the diamond particles or attaching to the diamond particles at least one material selected from the group consisting of biological molecules, a site-specific targeting ligand, a nucleic acid, a peptide, a protein, an antibody, an antigen, oligonucleotide, aptamer, RNA, DNA, a ligand, a dye, a fluorescent specie, a spin trap, a radioactive specie, an image contrast agent, an isotope, a drug molecule, a hormone, a carbohydrate, and a polymer.

43. The method according to claims 7, 17 or 18, where analyzed mixture comprises at least one of: a continuous flow in a capillary or a channel, flow in a microfluidic device, and content of a multi-well plate.

44. The method according to claims 7, 17 or 18, further comprising a microfluidic flow assay, a microplate reader, flow cytometry assay, fluorescence activated cell sorting, an ABEL trap, an acoustofluidic device, an electrophoretic device, a lateral flow assay, a vertical flow assay, PCR, and ELISA.

45. The method according to claims 7, 17 or 18, further comprising a lateral flow assay where:
a. fluorescent diamond particles are conjugated with a ligand that captures analyte;
b. fluorescent diamond particles with captured analyte are captured at a test line of a lateral flow strip; and
c. paramagnetic nature of analyte is concluded from analysis of the magnetically modulated fluorescence contrast of fluorescent diamond particles in step (b).

46. The method according to claim 45, where the analyte further comprises ferritin or apoferritin.

47. The method according to claims 7, 17 or 18, where diamond particles have size between approximately 5 nm and 1000 nm, preferably between 10 nm and 200 nm, and most preferably between 20 nm and 70 nm.

48. A device for detection of a chemical species comprising:
a. a container having a sample that contains an analyzed mixture that may contain the chemical species and fluorescent diamond particles containing at least a single NV center;
b. a light source for applying optical radiation with periodically time-varying intensity at an optical modulation frequency selected from about 10 Hz to about 10,000 Hz to the analyzed mixture;
c. a source generating a magnetic field for applying to the analyzed mixture at a magnetic modulation frequency selected from about 1 Hz to about 10,000 Hz;
d. a detector detecting fluorescence intensity from the analyzed mixture, where the fluorescence intensity is optically modulated, or optically and magnetically modulated; and
e. a signal processing device providing a phase-sensitive measurement of a detected fluorescence intensity modulated at two frequencies, where the detected fluorescence intensity is processed by at least one of: a dual-frequency lock-in amplifier, two individual lock-in amplifiers, digital lock-in analysis, computational lock-in analysis, Fourier analysis, analog-to-digital converter, and computer analyzer.

49. The method according to claims 18, or 48, where the magnetic modulation frequency is selected from about 10 Hz to about 1,000 Hz.
